(19) United States
(12) Patent Application Publication
(10) Pub. No.: US 2009/0028971 A1
(43) Pub. Date: Jan. 29, 2009

(54) Method for Obtaining Natural Oils From Plant Constituents

(75) Inventors: Johann Wiesmuller, Garching (DE); Stephan Pilz, Fellbach (DE)

Correspondence Address: FULBRIGHT & JAWORSKI, LLP, 666 FIFTH AVE, NEW YORK, NY, 10103-3198, US

(73) Assignee: DEGUSSA AG, Trostberg (DE)

(21) Appl. No.: 11/989,336

(22) PCT Filed: Aug. 4, 2006
(86) PCT No.: PCT/EP2006/007757

§ 371 Date: Jan. 24, 2008

(30) Foreign Application Priority Data
Aug. 6, 2005 (DE) 10 2005 037 209.0

(51) Int. Cl. A61K 36/00; C11B 1/10

(52) U.S. Cl. 424/776; 554/20; 554/12; 424/777

(57) ABSTRACT

The invention relates to a method for obtaining natural oils from plant constituents, said method being essentially carried out using compressed C₂-C₄ hydrocarbons. Residues from the crop and fruit treatment, especially from the treatment of pips and berries, are used as starting materials. Unlike previous extraction methods using organic solvents, the inventive method can be economically carried out on the basis of low pressures and reduced extraction agent throughputs. Preferred extraction agents are ethane, propane, butane and the mixtures thereof, the extraction itself being carried out in batches at pressures of <50 mPa and temperatures of ≦70° C., with an extraction agent throughput of between 4 and 20 kg/kg of starting materials. The extracted oils are outstandingly suitable for flavouring preparations and cosmetic formulations, especially for the care and treatment of the skin.

The present invention relates to a method for obtaining natural oils from plant constituents by extraction with the help of compressed gases.

Natural oils are increasingly important starting materials for the production of protection and care products in the cosmetics industry. They are also being used more in the health sector, one example being the treatment of allergies. The advantage of natural oils is in particular to be regarded as being the fact that they are ingredients of renewable raw materials which, from ecological and economical points of view, makes them appear extremely valuable. In addition, the use of natural ingredients significantly increases consumer acceptance since consumers have in the meantime high ecological awareness and select products for bodycare and healthcare extremely critically.

Various terms are used specifically in the area of natural oils, with oils being understood, according to general understanding, as meaning lipophilic substance mixtures of (semi-)liquid consistency. Natural oils of plant origin are generally mixtures of phytohormones, isoflavonoids, phytosterols, terpenoids, lipophilic vitamins (tocopherols) and short-chain fatty acid esters. These oils are thus typical natural products. Strict delimitation of the plant oils on the basis of the components involved in their composition can only be undertaken to a limited degree and is also not required in most cases.

Plant oils and in particular apple oils are already used by the cosmetics industry in diverse areas of application and in particular in skincare and haircare products.

The raw material source of these exemplary apple oils is primarily pomaces, i.e. more or less dried residues from the manufacture of apple juice and pectin. The apple oils present in this pomace originate in most cases from the pips of juice apples. After firstly the pomace has been sieved off to obtain the pips and the fruit waxes have been separated off from the remaining residue, the apple oil is generally obtained from the pressed apple pips with the help of a solvent. This raw material is accessible without further-reaching transformation, i.e. it can be purified and isolated by purely physical methods such as, for example, adsorption, filtration and distillation. The residue from the pip oil isolation can, like usually the total pomace, be used as cattle feed.

The already discussed isolation of the pip oils has thus hitherto taken place primarily with the help of organic solvents which are approved for the treatment of food. Since the cosmetics industry is increasingly demanding, irrespective of the particular field of use, purer products with a defined component spectrum, extraction with organic solvents, however, is no longer adequate, particularly including from economical points of view.

As already indicated, natural oils are not a single substance class, but a mixture of different substance classes. On account of the predominantly lipophilic composition of the pressed pips used hitherto for obtaining pip oil and the known risk-free properties of carbon dioxide, the use of supercritical CO₂ would be the method of choice. However, the disadvantageous process conditions such as high pressures and a high CO₂ throughput as well as low selectivity have led to disappointing results.

The European patent EP 591 981 B1 has already disclosed a method for the extraction of fats and oils from natural substances which is carried out with the help of liquid propane as solvent and in which recourse is made to fat- or oil-containing natural substances, including those based on plants, as starting material. Given examples of plant oils are olive oil, palm oil, rapeseed oil, flax oil and oil from sunflowers, wheat and rice germ, and also cotton seeds, soybean and palm kernels. The extraction carried out in this regard with liquid propane is carried out in a very narrow and defined pressure and temperature range, which is 10 to 30 bar and 10 to 55° C., since at pressures >30 bar, particularly in the case of the extraction of oil seeds, the extraction properties of the liquid propane become increasingly unselective. In the method described in this patent, plant natural substances are thus extracted which are typical sources of plant oils, such as olives, palm kernels, rapeseed, flax, sunflower kernels and wheat germs and rice germs.

In this European patent EP 591 981 B1, reference is also made to further prior art documents which previously describe the use of compressed propane for the extraction of oils. According to DE-A 28 43 920, crude plant fats and oils are refined with supercritical gases such as propane and CO₂. According to U.S. Pat. No. 2,254,245, a fat extraction takes place at very low temperatures <0° C., whereas according to U.S. Pat. No. 1,802,533, a maximum extraction pressure of 7 bar is recommended. Furthermore, reference is also made to the fact that critical state parameters are often selected when separating off the extracted lipids since, in this way, it is possible to utilize a phase separation into an oil-rich and a low-oil propane phase in order, in so doing, to separate off or to fractionate the oil (U.S. Pat. No. 2,660,590 or U.S. Pat. No. 2,548,434).

All of the cited methods have recourse to starting materials which are typical sources of natural oils, it being pointed out time and again that the particular process parameters in the case of the use of compressed hydrocarbons, such as, for example, propane, are to be selected such that a maximum selectivity with regard to the oils to be extracted is achieved. The isolation of plant oils from nonspecific plant starting materials has hitherto not been described previously.

On account of this disadvantage of the prior art, the object set for the present invention is to provide a novel method for obtaining natural oils from plant constituents by extraction with the help of compressed C₂-C₄-hydrocarbons which meets the increased requirements by the processing industry and by the consumer. The novel method should be commercially implementable in the simplest possible manner and provide natural oils from atypical starting materials in improved quality.

This object was achieved with a method which, with regard to the starting material, has recourse to residues from the processing of fruit, where in particular residues from the processing of fruit pips and berries and particularly preferably from the production of juice are suitable. In particular, residues from apples, pears, citrus fruits, such as mandarins, oranges or lemons, melons, pumpkins, raspberries, blackberries, elderberries and/or currants are suitable as starting materials, where the residues should in particular have large contents of pips, e.g. contents of ≧50% by weight and in particular ≧90% by weight. The water content of the extraction material is preferably <15% by weight and in particular <10% by weight.

Suitable starting materials are, however, also pieces of rind, leaves, stems and woody tissue of plant parts above and below the ground, which can all have oil cells. These residues merely have to be precomminuted and possibly washed.

In practice, it has been found that with this novel method according to the invention, not only could the objective be achieved but that natural oils and in particular pip oils can surprisingly be obtained from plant residues in sometimes significantly increased yields. Although these yields do not regularly exceed the yields as are obtainable with the help of organic solvents according to the prior art, the oils obtainable according to the invention have a qualitatively superior content spectrum. The oils obtainable with the method according to the invention are overall of a quality which renders them accessible to new fields of application.

As extraction agent, the present invention considers ethane, propane and butane, and mixtures thereof. In this connection, if necessary, entrainers such as dimethyl ether and alcohols can also be added to the compressed hydrocarbon (mixture) used in each case as extraction agent, in which case these are then preferably used in amounts of from 0.5 to 50% by weight.

As regards the extraction parameters, the present invention recommends pressures of <50 MPa and temperatures of ≦70° C., with a pressure range between 0.5 and 10 MPa and temperatures between 20 and 35° C. being regarded as particularly preferred.

Depending on the particular starting material (or its moisture content), the method according to the present invention should be carried out with an extraction agent throughput of from 4 to 20 kg/kg of starting material, with a throughput of between 5 and 10 kg/kg of starting material being regarded as preferred.

The extracts in each case are usually separated off by a pressure drop to 5 to 10 bar at temperatures between t 42 and 48° C.

Although the proposed method can also be carried out continuously, for practical reasons, the invention envisages carrying out the method batchwise since, in so doing, the process parameters can be matched to the particular starting material, its nature and composition in a readily comprehensible manner.

Besides the actual method itself, the present invention also claims the use of a plant oil extracted using the proposed method in cosmetic preparations and in particular as aroma substance or for the care and treatment of the skin and appendages thereof, which is to be understood primarily as meaning hair and toe and finger nails. Within the scope of the present invention, the term “skin” is naturally to be understood firstly as meaning the skin itself, but also mucosae and the skin appendages, where these comprise living cells. Here, in particular hair follicle, hair root, hair bulb and the ventral epithelium of the nail bed, sebaceous glands and sweat glands are to be understood. The use according to the invention includes the care and also therapeutic and nontherapetuic treatment, which includes in particular aroma therapy.

With the novel method for the extraction of natural oils from plant constituents with the help of C₂-C₄-hydrocarbons, it is possible to obtain natural oils in improved quality and increased yields under extremely economical conditions from starting materials which are usually typical waste materials and are now used as secondary raw materials.

The example below demonstrates the advantages of the claimed method.

### EXAMPLE

In the production of raspberry juice, pips are typically produced as sieve residue; these contain natural fruit and aroma oils.

These pips were freed from remaining fruit flesh, dried and finally ground. The comminuted pips are then extracted in an autoclave at 30 bar and 35° C. with liquid propane, the extraction agent throughput being on average 6.8 kg/kg of starting material. The extract obtained in this way was separated off after the pressure had been reduced at 8 bar and 46° C.

In this way, a yellow-green and clear raspberry pip oil extract was obtained in a yield of about 14%.

1-10. (canceled)

11. A method for obtaining a plant oil from a plant constituent, comprising extracting said plant oil from said plant constituent with a compressed C₂-C₄ hydrocarbon, at a pressure of <50 MPa and a temperature of ≦70° C., wherein said plant constituent is a residue obtained from processes of fruit pips or berries.

12. The method of claim 11, wherein said plant constituent is a by product of juice production.

13. The method of claim 11, wherein said plant constituent comprises plant pips.

14. The method of claim 11, wherein said residue is a residue of an apple, a pear, a citrus fruit, a melon, a pumpkin, a raspberry, a blackberry, an elderberry, or a currant.

15. The method of claim 14, wherein said citrus fruit is a mandarin, an orange or a lemon.

16. The method of claim 14, wherein said residue is a residue of an apple.

17. The method of claim 14, wherein said residue is a residue of a raspberry.

18. The method of claim 11, comprising extracting with ethane, butane, propane, or a mixture of at least two of these.

19. The method of claim 11, further comprising adjusting said pressure to 0.5 to 10 MPa and adjusting said temperature to 20° C. to 35° C.

20. The method of claim 11, further comprising adding an entrainer to said compressed, C₂-C₄ hydrocarbon.

21. The method of claim 20, wherein said entrainer is dimethyl ether or an alcohol.

22. The method of claim 21, wherein said entrainer is added in an amount ranging from 0.5% to 50% by weight to said compressed, C₂-C₄ hydrocarbon mixture.

23. The method of claim 11, wherein said method comprises extracting with an extraction agent throughput of from 4 kg/kg to 20 kg/kg.

24. The method of claim 23, wherein said throughput is from 5 kg/kg to 10 kg/kg.

25. The method of claim 11, carried out batchwise.

26. A cosmetic preparation comprising the plant oil produced by claim 11 and a pharmaceutically acceptable carrier.
